(19) United States
(12) Patent Application Publication
(10) Pub. No.: US 2026/0159623 A1
(43) Pub. Date: Jun. 11, 2026

(54) PHOTOCURABLE COMPOSITION

(71) Applicant: THREEBOND CO., LTD., Hachioji-shi, Tokyo (JP)

(72) Inventor: Natsumi HARADA, Hachioji-shi, Tokyo (JP)

(21) Appl. No.: 18/707,270

(22) PCT Filed: Oct. 31, 2022
(86) PCT No.: PCT/JP2022/040730

§ 371 Date: May 3, 2024

(30) Foreign Application Priority Data
Nov. 12, 2021 (JP) 2021-184528

(51) Int. Cl. C08F 283/00; A45D 31/00; A61K 8/46; A61K 8/91; A61Q 3/02; C08F 2/50; C08K 5/07; C08K 5/3475; C08K 5/3492; C08K 5/37

(52) U.S. Cl. CPC C08F 283/008; A45D 31/00; A61K 8/46; A61K 8/91; A61Q 3/02; C08F 2/50; C08K 5/07; C08K 5/3475; C08K 5/3492; C08K 5/37; A61K 2800/52; A61K 2800/81

(57) ABSTRACT

A photocurable composition contains component (A) to component (D): Component (A): a compound having a (meth)acryloyl group; Component (B): a polythiol compound; Component (C): a photoinitiator; and Component (D): a phenol compound having one phenol group in its molecule, the compound having a specific structure, except for the component (A).

### TECHNICAL FIELD

The present invention relates to a photocurable composition containing a polythiol compound.

### BACKGROUND ART

It is known that surface curability is enhanced by addition of a polythiol compound to a compound having a (meth)acryloyl group. The reactivity is basically high and thus heat generation occurs in curing. In general, use of a polyfunctional compound having a plurality of (meth)acryloyl groups in its molecule leads to further heat generation. Japanese Patent Laid-Open No. 2019-034897 recites the invention relating to a so-called gel nail, and procedures are applied to a subject by a manicurist. In such procedures, a nail or an artificial nail is coated with a gel nail and then subjected to light irradiation for curing. Heat generation often occurs in photocuring, and it is known that a large amount of a compound having only one (meth)acryloyl group in its molecule (monofunctional) is added to a (meth)acrylate monomer in order to suppress heat generation. However, durability of a cured product tends to be reduced.

### SUMMARY OF INVENTION

It has been conventionally difficult to suppress heat generation in photocuring of a photocurable composition of a compound having a (meth)acryloyl group, containing a polythiol compound.

An object of the present invention to provide a means for enabling a photocurable composition of a compound having a (meth)acryloyl group, containing a polythiol compound, to be cured with suppression of heat generation in photocuring even in the case of the composition containing a compound having two or more (meth)acryloyl groups in its molecule.

The present inventors have made intensive studies in order to achieve the above object, and as a result, have completed the present invention relating to the following photocurable composition.

In a case where a range of an upper limit value to a lower limit value is expressed with “-”, the range is intended to also include numerical values of the lower limit value and the upper limit value.

The gist of the present invention is then described. A first aspect of the present invention is a photocurable composition containing components (A) to (D):
Component (A): a compound having a (meth)acryloyl group
Component (B): a polythiol compound
Component (C): a photoinitiator
Component (D): a phenol compound having one phenol group in its molecule, represented by the following general formula 1 (except for the component (A)).

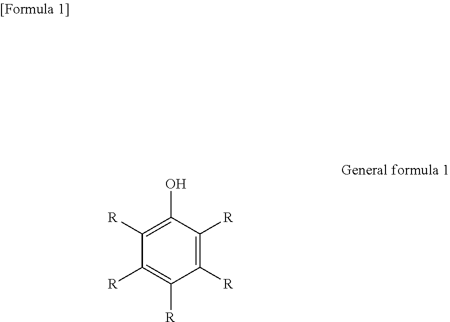

Herein, only one among Rs is an organic group conjugated with a phenol aromatic ring, and the remaining Rs are each independently hydrogen or a linear organic group.

A second aspect of the present invention is the photocurable composition according to the first aspect, wherein the organic group conjugated with a phenol aromatic ring is a group of formula 2, a group of formula 3 or a group of formula 4, or a group having a backbone of formula 2, formula 3 or formula 4, described below.

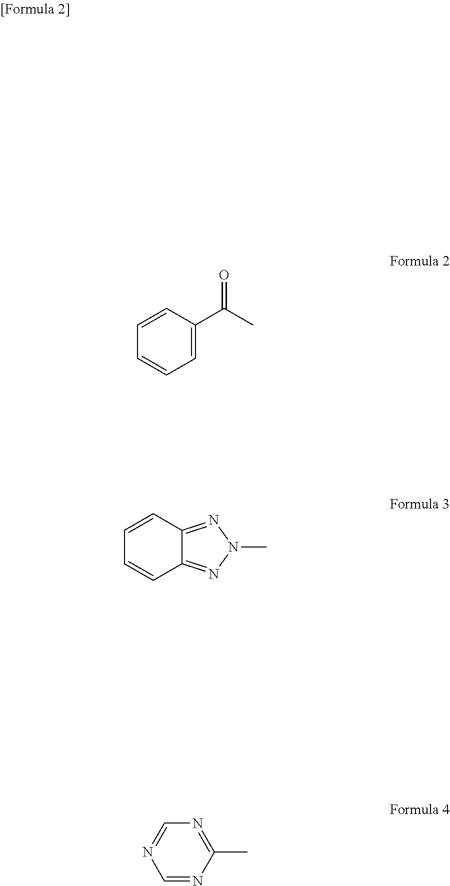

A third aspect of the present invention is the photocurable composition according to the first aspect or the second aspect, the composition containing 0.001 to 10.0% by mass of the component (D) relative to the entire photocurable composition.

A fourth aspect of the present invention is the photocurable composition according to any of the first aspect to the third aspect, the composition containing no ultraviolet absorber other than the component (D) in the photocurable composition.

A fifth aspect of the present invention is the photocurable composition according to any of the first aspect to the fourth aspect, the composition containing 0.1 to 50 parts by mass of the component (B) and 0.1 to 10 parts by mass of the component (C) based on 100 parts by mass of the component (A).

A sixth aspect of the present invention is the photocurable composition according to any of the first aspect to the fifth aspect, wherein the component (A) is composed of a (meth)acrylate oligomer and a (meth)acrylate monomer.

A seventh aspect of the present invention is the photocurable composition according to the sixth aspect, the composition containing a (meth)acrylate oligomer having 3 to 5 (meth)acryloyl groups per molecule, as the (meth)acrylate oligomer.

An eighth aspect of the present invention is the photocurable composition according to the sixth aspect or the seventh aspect, wherein the (meth)acrylate monomer contains tricyclodecane dimethanol di(meth)acrylate.

A ninth aspect of the present invention is the photocurable composition according to any of the first aspect to the eighth aspect, for use in a nail or an artificial nail.

A tenth aspect of the present invention is the photocurable composition according to the ninth aspect, wherein the composition is for a topcoat.

### DESCRIPTION OF EMBODIMENTS

One aspect of the present invention relates to a photocurable composition containing component (A) to component (D):
Component (A): a compound having a (meth)acryloyl group;
Component (B): a polythiol compound;
Component (C): a photoinitiator; and
Component (D): a phenol compound having one phenol group in its molecule, of the general formula 1 (except for the component (A)).

In the present invention, a photocurable composition of a compound having a (meth)acryloyl group, containing a polythiol compound, can be cured with suppression of heat generation in photocuring, even when contains a compound having two or more (meth)acryloyl groups in its molecule.

The detail of the present invention is then described. The component (A) usable in the present invention can be any compound having a (meth)acrylic group, specifically, refers to a compound such as (meth)acrylate or (meth)acrylamide, and also encompasses a (meth)acrylate monomer and a (meth)acrylate oligomer. The component (A) is preferably composed of a (meth)acrylate oligomer and a (meth)acrylate monomer. In the present invention, “acrylic” and “methacrylic” are collectively called “(meth)acrylic”. The component (A) is preferably in the form of a liquid under an atmosphere at 25° C., and can be used as long as it has favorable compatibility with the following component (B) and component (C) according to the present invention.

Specific examples of the (meth)acrylate oligomer include a (meth)acrylate oligomer having an ester bond in its molecule, a (meth)acrylate oligomer having an ether bond in its molecule, a (meth)acrylate oligomer having a urethane bond in its molecule, and an epoxy-modified (meth)acrylate oligomer in its molecule, and examples of the main backbone thereof include bisphenol A, novolac phenol, polybutadiene, polyester, and polyether, but not limited thereto. The component (A) usable in the present invention also encompasses a compound having one or more epoxy groups and one or more acrylic groups in one molecule. The photocurable composition preferably contains a (meth)acrylate oligomer having 3 to 5 (meth)acryloyl groups in one molecule from the viewpoints of an enhancement in photocurability and a reduction in heat generation during procedures.

A known method for synthesizing the (meth)acrylate oligomer having an ester bond is, for example, a synthesis method including forming an ester bond by a polyol and a polyvalent carboxylic acid and adding acrylic acid to an unreacted hydroxyl group, but not limited to this synthesis method. Specific examples of the (meth)acrylate oligomer having an ester bond include Aronix M-6100, M-6200, M-6250, M-6500, M-7100, M-7300K, M-8030, M-8060, M-8100, M-8530, M-8560, and M-9050 manufactured by Toagosei Co., Ltd., and UV-3500BA, UV-3520TL, UV-3200B, and UV-3000B manufactured by Nippon Synthetic Chemical Industry Co., Ltd., but not limited thereto.

A known method for synthesizing the (meth)acrylate oligomer having an ether bond is, for example, a synthesis method including adding acrylic acid to a hydroxyl group of a polyether polyol, or a hydroxyl group of an aromatic polyether polyol such as bisphenol, but not limited to this synthesis method. Specific examples of the (meth)acrylate oligomer having an ether bond include UV-6640B, UV-6100B, UV-3700B, and the like manufactured by Nippon Synthetic Chemical Industry Co., Ltd., Light (meth)acrylates 3EG-A, 4EG-A, 9EG-A, 14EG-A, PTMGA-250, BP-4EA, BP-4PA, BP-10EA, and the like manufactured by Kyoeisha Chemical Co., Ltd., and EBECRYL3700 manufactured by Daicel-Cytec Co., Ltd., but not limited thereto.

A known method for synthesizing the (meth)acrylate oligomer having a urethane bond is, for example, a synthesis method including forming a urethane bond by a polyol and a polyisocyanate and adding a compound having a hydroxyl group and a (meth)acrylic group in its molecule, or (meth)acrylic acid to the remaining isocyanate group, but not limited to this synthesis method. Examples of the polyol include polyether polyol, polyester polyol, and polycarbonate polyol, but not limited thereto. The component (A) added is preferably the (meth)acrylate oligomer having a urethane bond, most preferably a (meth)acrylate oligomer having a polyether backbone and having a urethane bond, from the viewpoint of an enhancement in durability. Specific examples of the (meth)acrylate oligomer having a urethane bond include KY-11, UN-904, UN-3320HA, and KY-103 manufactured by Negami Chemical Industrial Co., Ltd., and AH-600, AT-600, UA-306H, and UF-8001G manufactured by Kyoeisha Chemical Co., Ltd., but not limited thereto.

A known method for synthesizing the epoxy-modified (meth)acrylate oligomer is, for example, a synthesis method including subjecting a glycidyl group of a polyfunctional glycidyl ether compound to ring-opening polymerization with (meth)acrylic acid or the like, but not limited to this synthesis method. Various types of backbones such as a bisphenol A type, a bisphenol F type, and a novolac phenol type can be used in a main chain of such a polyfunctional glycidyl ether. Specific examples of the epoxy-modified acrylic oligomer include epoxy esters 3000A and 3002A manufactured by Kyoeisha Chemical Co., Ltd., and EBECRYL3700 manufactured by Daicel-Allnex Ltd., but not limited thereto.

The weight average molecular weight (or molecular weight) of the (meth)acrylate oligomer is preferably 1000 to 50000. A weight average molecular weight of 1000 or more allows a cured product to exhibit toughness, and a weight average molecular weight of 50000 or less can allow the viscosity of the composition to be kept low.

The (meth)acrylate monomer can include monofunctional, difunctional, or trifunctional (meth)acrylate monomer and (meth)acrylamide monomer, and the (meth)acrylate monomer can be used singly or in combinations of a plurality thereof.

Specific examples of the monofunctional (meth)acrylate monomer include lauryl (meth)acrylate, stearyl (meth)acrylate, ethyl carbitol (meth)acrylate, tetrahydrofurfuryl (meth)acrylate, caprolactone-modified tetrahydrofurfuryl (meth)acrylate, cyclohexyl (meth)acrylate, dicyclopentanyl (meth)acrylate, isobornyl (meth)acrylate, benzyl (meth)acrylate, phenyl (meth)acrylate, phenoxyethyl (meth)acrylate, phenoxydiethylene glycol (meth)acrylate, phenoxytetraethylene glycol (meth)acrylate, nonylphenoxyethyl (meth)acrylate, nonylphenoxytetraethylene glycol (meth)acrylate, methoxydiethylene glycol (meth)acrylate, ethoxydiethylene glycol (meth)acrylate, butoxyethyl (meth)acrylate, butoxytriethylene glycol (meth)acrylate, 2-ethylhexylpolyethylene glycol (meth)acrylate, 4-hydroxybutyl (meth)acrylate, nonylphenylpolypropylene glycol (meth)acrylate, methoxydipropylene glycol (meth)acrylate, 2-hydroxyethyl (meth)acrylate, 2-hydroxypropyl (meth)acrylate, glycerol (meth)acrylate, polyethylene glycol (meth)acrylate, polypropylene glycol (meth)acrylate, epichlorohydrin-modified butyl (meth)acrylate, epichlorohydrin-modified phenoxy (meth)acrylate, N, N-dimethylaminoethyl (meth)acrylate, and N, N-diethylaminoethyl (meth)acrylate, but not limited thereto. The component (A) preferably contains a monofunctional (meth)acrylate having a hydroxyl group. Specific examples of the monofunctional (meth)acrylate having a hydroxyl group include 2-hydroxyethyl (meth)acrylate and 2-hydroxypropyl (meth)acrylate, but not limited thereto.

Examples of the monofunctional (meth)acrylate monomer also include a (meth)acrylate monomer having an acidic group. Examples of the (meth)acrylate monomer having an acidic group particularly include carboxylic acid or phosphoric acid having a (meth)acrylic group in its molecule. Examples of the carboxylic acid having a (meth)acrylic group in its molecule include (meth)acrylic acid, 3-(meth)acryloyloxypropylsuccinic acid, 4-(meth)acryloyloxybutylsuccinic acid, 2-(meth)acryloyloxyethylmaleic acid, 3-(meth)acryloyloxypropylmaleic acid, 4-(meth)acryloyloxybutylmaleic acid, 2-(meth)acryloyloxyethylhexahydrophthalic acid, 3-(meth)acryloyloxypropylhexahydrophthalic acid, 4-(meth)acryloyloxybutylhexahydrophthalic acid, 2-(meth)acryloyloxyethylphthalic acid, 3-(meth)acryloyloxypropylphthalic acid, and 4-(meth)acryloyloxybutylphthalic acid, but not limited thereto. Examples of the phosphoric acid having a (meth)acrylic group in its molecule include 2-ethylhexyl acid phosphate, 2-hydroxyethyl methacrylate acid phosphate, and dibutyl phosphate, but not limited thereto. The component (A) preferably contains the (meth)acrylate monomer having an acidic group from the viewpoint of an enhancement in durability.

Specific examples of the difunctional (meth)acrylate monomer include 1,3-butylene glycol di(meth)acrylate, 1,4-butylene glycol di(meth)acrylate, tricyclodecane dimethanol di(meth)acrylate, neopentyl glycol di(meth)acrylate, 1,6-hexane glycol di(meth)acrylate, ethylene glycol diacrylate, tetraethylene glycol di(meth)acrylate, polyethylene glycol di(meth)acrylate, propylene glycol di(meth)acrylate, tripropylene glycol di(meth)acrylate, ethylene oxide-modified neopentyl glycol di(meth)acrylate, propylene oxide-modified neopentyl glycol di(meth)acrylate, bisphenol A di(meth)acrylate, ethylene oxide-modified bisphenol A di(meth)acrylate, epichlorohydrin-modified bisphenol A di(meth)acrylate, ethylene oxide-modified bisphenol S di(meth)acrylate, neopentyl glycol-modified trimethylolpropane di(meth)acrylate, dicyclopentenyl di(meth)acrylate, ethylene oxide-modified dicyclopentenyl di(meth)acrylate, and diacryloyl isocyanurate, but not limited thereto. The component (A) preferably contains tricyclodecane dimethanol di(meth)acrylate particularly from the viewpoint of surface curability.

Specific examples of the trifunctional (meth)acrylate monomer include trimethylolpropane tri(meth)acrylate, trimethylolpropane tri(meth)acrylate, pentaerythritol tri(meth)acrylate, EO-modified trimethylolpropane tri(meth)acrylate, PO-modified trimethylolpropane tri(meth)acrylate, ECH-modified trimethylolpropane tri(meth)acrylate, ECH-modified glycerol tri(meth)acrylate, and tris(acryloyloxyethyl) isocyanurate, but not limited thereto.

Specific examples of the (meth)acrylamide monomer include dimethyl(meth)acrylamide, (meth)acryloylmorpholine, and diethyl(meth)acrylamide, but not limited thereto. Although no clear reason is found, the (meth)acrylamide monomer preferably contains the (meth)acrylamide monomer from the viewpoint of an enhancement in durability. DMAA, ACMO, DEAA, and the like manufactured by KJ Chemicals Corporation are known as specific examples of the (meth)acrylamide monomer in the present invention, without limitation thereto.

The component (A) preferably contains both the (meth)acrylate oligomer and the (meth)acrylate monomer, and the ratio of the oligomer to the monomer (mass ratio) is preferably 50:50 to 95:5. The component (A) contains the (meth)acrylate oligomer to allow an enhancement in durability to be exhibited.

The component (B) usable in the present invention is a polythiol compound. The component (B) is not particularly limited as long as it has two or more thiol groups, and may be used singly or in combinations of two or more kinds thereof. Specific examples of the component (B) include an aliphatic polythiol compound, an aromatic polythiol compound, and a polythiol compound having a sulfide bond, but not limited thereto.

Examples of an aliphatic polythiol compound having two thiol groups include 1,2-ethanedithiol, 1,2-propanedithiol, 1,3-propanedithiol, 1,4-butanedithiol, 1,6-hexanedithiol, 1,7-heptanedithiol, 1,8-octanedithiol, 1,9-nonanedithiol, 1,10-decanedithiol, 1,12-dodecanedithiol, 2,2-dimethyl-1,3-propanedithiol, 3-methyl-1,5-pentanedithiol, 2-methyl-1,8-octanedithiol, 1,4-cyclohexanedithiol, 1,4-bis(mercaptomethyl)cyclohexane, 1,1-cyclohexanedithiol, 1,2-cyclohexanedithiol, bicyclo[2,2,1]hepta-exo-cis-2,3-dithiol, 1,1-bis(mercaptomethyl)cyclohexane, bis(2-mercaptoethyl) ether, ethylene glycol bis(2-mercaptoacetate), and ethylene glycol bis(3-mercaptopropionate), but not limited thereto.

Examples of an aliphatic polythiol compound having three thiol groups include 1,1,1-tris(mercaptomethyl) ethane, 2-ethyl-2-mercaptomethyl-1,3-propanedithiol, 1,2,3-propanetrithiol, trimethylolpropane tris(2-mercaptoacetate), trimethylolpropane tris(3-mercaptopropionate), and tris[(mercaptopropynyloxy)-ethyl]isocyanurate, but not limited thereto.

Examples of an aliphatic polythiol compound having four or more thiol groups include pentaerythritol tetrakis(2-mercaptoacetate), pentaerythritol tetrakis(3-mercaptopropionate), pentaerythritol tetrakis(3-mercaptobutyrate), and dipentaerythritol hexa-3-mercaptopropionate, but not limited thereto.

Examples of the aromatic polythiol compound include 1,2-dimercaptobenzene, 1,3-dimercaptobenzene, 1,4-dimercaptobenzene, 1,2-bis(mercaptomethyl)benzene, 1,3-bis(mercaptomethyl)benzene, 1,4-bis(mercaptomethyl)benzene, 1,2-bis(2-mercaptoethyl)benzene, 1,3-bis(2-mercaptoethyl)benzene, 1,4-bis(2-mercaptoethyl)benzene, 1,2-bis(2-mercaptoethyleneoxy)benzene, 1,3-bis(2-mercaptoethyleneoxy)benzene, 1,4-bis(2-mercaptoethyleneoxy)benzene, 1,2,3-trimercaptobenzene, 1,2,4-trimercaptobenzene, 1,3,5-trimercaptobenzene, 1,2,3-tris(mercaptomethyl)benzene, 1,2,4-tris(mercaptomethyl)benzene, 1,3,5-tris(mercaptomethyl)benzene, 1,2,3-tris(2-mercaptoethyl)benzene, 1,2,4-tris(2-mercaptoethyl)benzene, 1,3,5-tris(2-mercaptoethyl)benzene, 1,2,3-tris(2-mercaptoethyleneoxy)benzene, 1,2,4-tris(2-mercaptoethyleneoxy)benzene, 1,3,5-tris(2-mercaptoethyleneoxy)benzene, 1,2,3,4-tetramercaptobenzene, 1,2,3,5-tetramercaptobenzene, 1,2,4,5-tetramercaptobenzene, 1,2,3,4-tetrakis(mercaptomethyl)benzene, 1,2,3,5-tetrakis(mercaptomethyl)benzene, 1,2,4,5-tetrakis(mercaptomethyl)benzene, 1,2,3,4-tetrakis(2-mercaptoethyl)benzene, 1,2,3,5-tetrakis(2-mercaptoethyl)benzene, 1,2,4,5-tetrakis(2-mercaptoethyl)benzene, 1,2,3,4-tetrakis(2-mercaptoethyleneoxy)benzene, 1,2,3,5-tetrakis(2-mercaptoethyleneoxy)benzene, 1,2,4,5-tetrakis(2-mercaptoethyleneoxy)benzene, 2,2′-mercaptobiphenyl, 4,4′-thiobis-benzenethiol, 4,4′-dimercaptobiphenyl, 4,4′-dimercaptobibenzyl, 2,5-toluenedithiol, 3,4-toluenedithiol, 1,4-naphthalenedithiol, 1,5-naphthalenedithiol, 2,6-naphthalenedithiol, 2,7-naphthalenedithiol, 2,4-dimethylbenzene-1,3-dithiol, 4,5-dimethylbenzene-1,3-dithiol, 9,10-anthracenedimethanethiol, 1,3-bis(2-mercaptoethylthio)benzene, 1,4-bis(2-mercaptoethylthio)benzene, 1,2-bis(2-mercaptoethylthiomethyl)benzene, 1,3-bis(2-mercaptoethylthiomethyl)benzene, 1,4-bis(2-mercaptoethylthiomethyl)benzene, 1,2,3-tris(2-mercaptoethylthio)benzene, 1,2,4-tris(2-mercaptoethylthio)benzene, 1,3,5-tris(2-mercaptoethylthio)benzene, 1,2,3,4-tetrakis(2-mercaptoethylthio)benzene, 1,2,3,5-tetrakis(2-mercaptoethylthio)benzene, and 1,2,4,5-tetrakis(2-mercaptoethylthio)benzene, but not limited thereto.

Examples of the polythiol compound having a sulfide bond include bis(2-mercaptoethyl)sulfide, bis(2-mercaptoethylthio)methane, 1,2-bis(2-mercaptoethylthio)ethane, 1,3-bis(2-mercaptoethylthio)propane, 1,2,3-tris(2-mercaptoethylthio)propane, tetrakis(2-mercaptoethylthiomethyl)methane, 1,2-bis(2-mercaptoethylthio)propanethiol, 2,5-dimercapto-1,4-dithiane, bis(2-mercaptoethyl)disulfide, 3,4-thiophenedithiol, 1,2-bis(2-mercaptoethyl)thio-3-mercaptopropane, and bis-(2-mercaptoethylthio-3-mercaptopropane) sulfide, but not limited thereto.

Specific examples of the component (B) having two or more thiol groups include pentaerythritol tetrakis(3-mercaptobutyrate), 1,4-bis(3-mercaptobutyryloxy) butane, 1,3,5-tris(3-mercaptobutyryloxyethyl)-1,3,5-triazin-2,4,6 (1H, 3H, 5H)-trione, trimethylolpropane tris(3-mercaptobutyrate), trimethylolethane tris(3-mercaptobutyrate), trimethylolpropane tris(3-mercaptobutyrate), and trimethylolethane tris(3-mercaptobutyrate), but not limited thereto. Examples of a commercial product include PEMP and TMMP-20P manufactured by SC Organic Chemical Co., Ltd., and KarenzMT (registered trademark) series, PE1, BD1, and NR1 manufactured by Showa Denko K.K., but not limited thereto.

The amount of the component (B) added based on 100 parts by mass of the component (A) is preferably 0.1 to 50 parts by mass, particularly preferably 1 to 30 parts by mass. The amount of the component (B) contained is 0.1 parts by mass or more to result in an enhancement in surface curability, and the amount contained is 50 parts by mass or less to result in an enhancement in storage stability.

The component (C) usable in the present invention is a photoinitiator. The component (C) is not limited as long as it is a radical photoinitiator which generates radical species by an energy ray such as visible light, ultraviolet light, X-ray, or electron beam. The photoinitiator may be a non-visible light type photoinitiator or may be a visible light type photoinitiator.

Specific examples of the component (C) include acetophenones such as diethoxyacetophenone, 2-hydroxy-2-methyl-1-phenylpropan-1-one, benzyl dimethyl ketal, 4-(2-hydroxyethoxy)phenyl-(2-hydroxy-2-propyl) ketone, 1-hydroxycyclohexyl phenyl ketone, 2-methyl-2-morpholino (4-thiomethylphenyl)propan-1-one, 2-benzyl-2-dimethylamino-1-(4-morpholino phenyl) butanone, and a 2-hydroxy-2-methyl-1-[4-(1-methylvinyl)phenyl]propanone oligomer; benzoins such as benzoin, benzoin methyl ether, benzoin ethyl ether, benzoin isopropyl ether, and benzoin isobutyl ether; benzophenones such as benzophenone, methyl o-benzoylbenzoate, 4-phenylbenzophenone, 4-benzoyl-4′-methyl-diphenyl sulfide, 3,3′,4,4′-tetra(t-butylperoxycarbonyl)benzophenone, 2,4,6-trimethylbenzophenone, 4-benzoyl-N, N-dimethyl-N-[2-(1-oxo-2-propenyloxy)ethyl]benzenemethanaminium bromide, and (4-benzoylbenzyl)trimethylammonium chloride; and thioxanthones such as 2-isopropylthioxanthone, 4-isopropylthioxanthone, 2,4-diethylthioxanthone, 2,4-dichlorothioxanthone, 1-chloro-4-propoxythioxanthone, and 2-(3-dimethylamino-2-hydroxy)-3,4-dimethyl-9H-thioxanthon-9-one-mesochloride, but not limited thereto. The component (C) can also be used in combinations thereof.

The amount of the component (C) added based on 100 parts by mass of the component (A) is 0.1 to 20 parts by mass, further preferably 0.1 to 10 parts by mass. When the amount of the component (C) added is 0.1 parts by mass or more, photocurability can be maintained. On the other hand, when the amount of the component (C) added is 20 parts by mass or less, storage stability can be maintained without thickening during storage. The component (C) preferably contains 0 to 70% by weight of the visible light type photoinitiator relative to the entire component (C). Thus, yellowing of a cured product is hardly caused. The visible light type photoinitiator is here a photoinitiator which most strongly absorbs light in the visible region and which mainly represents an acyl phosphine oxide-based photopolymerization initiator containing a phosphorus atom. Specific examples of the visible light type photoinitiator include 2,4,6-trimethylbenzoyl-diphenyl-phosphine oxide and bis(2,4,6-trimethylbenzoyl)-phenylphosphine oxide, but not limited thereto.

The component (D) usable in the present invention is a phenol compound having one phenol group in its molecule, represented by general formula 1, except for the component (A). Herein, only one among Rs is an organic group conjugated with a phenol aromatic ring, and the remaining Rs are each independently hydrogen or a linear organic group. The linear organic group contains neither an aromatic ring, nor an alicyclic structure. Examples of the linear organic group include a hydrocarbon group, and a hydrocarbon group containing an oxygen atom. Examples of the hydrocarbon group include a hydrocarbon group having 1 to 20 carbon atoms. Examples of the hydrocarbon group containing an oxygen atom include a group in which one or more hydrogen atoms in a hydrocarbon group are each substituted with a group containing an oxygen atom (for example, an oxygen atom, a hydroxy group, or a carboxy group), and a group in which one or more carbon atoms in a hydrocarbon group are each substituted with an oxygen atom. The molecular weight of the component (D) is preferably 150 to 600 from the viewpoints of solubility in the component (A) and the component (B). The component (D) may be used as an ultraviolet absorber, and the effect of suppressing heat generation in curing has been confirmed although no clear reason is found in the present invention. The composition preferably contains no ultraviolet absorber other than the component (D) in order to allow for no reduction in effect of suppression of heat generation.

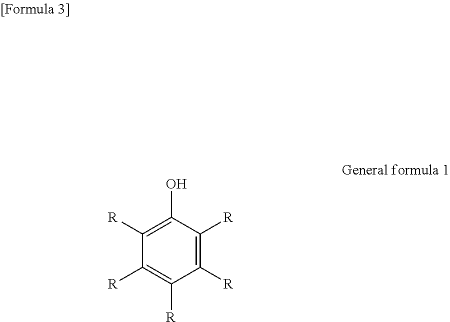

The organic group conjugated with a phenol aromatic ring is preferably a group of formula 2, a group of formula 3 or a group of formula 4, or a group having a backbone of formula 2, formula 3 or formula 4.

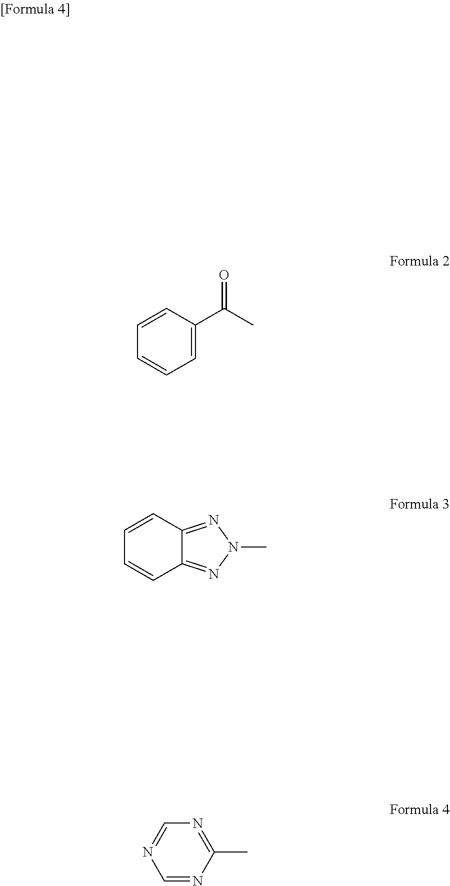

Specific examples of the component (D) include [2-hydroxy-4-(octyloxy)phenyl](phenyl)methanone, 2-(4,6-diphenyl-1,3,5-triazin-2-yl)-5-[2-(2-ethylhexanolyoxy)ethoxy]phenol, 2-(2H-benzotriazol-2-yl)-4-(1,1,3,3-tetramethylbutyl) phenol, 2-(2H-benzotriazol-2-yl)-p-cresol, and 2-(5-chloro-2H-benzotriazol-2-yl)-6-tert-butyl-4-methylphenol, but not limited thereto.

The amount of the component (D) added based on 100 parts by mass of the component (A) is preferably 0.01 to 5.0 parts by mass. In a case where the amount of the component (D) added is 0.01 parts by mass or more, heat generation in photocuring can be suppressed, and in a case where the amount is 5.0 parts by mass or less, photocurability, particularly, surface curability can be maintained. The amount of the component (D) contained is preferably 0.001 to 10.0% by mass relative to the entire photocurable composition of the present invention. When the amount of the component (D) contained is 0.001% by mass or more, heat generation can be suppressed, and the amount contained is 10.0% by mass or less, storage stability can be kept good.

A proper amount of additive(s) such as a coupling agent, an inorganic filling agent or an organic filling agent, a colorant including a pigment, a dye and the like, an antioxidant, a polymerization inhibitor, a defoaming agent, a leveling agent, and/or a rheology control agent may be compounded in the photocurable composition of the present invention, as long as features of the present invention are not impaired. In one embodiment of the present invention, a leveling agent is compounded. Such addition provides a composition excellent in resin strength, adhesion strength, workability, storage stability, and the like, or a cured product thereof.

The leveling agent is preferably one containing a siloxane-based compound or a silicone resin in a component, more preferably one containing modified polyorganosiloxane therein. Examples include polyether-modified polyorganosiloxane, amine-modified polyorganosiloxane, polydimethylsiloxane, and a siloxane-modified copolymer, but not limited thereto.

Specific examples of the leveling agent, as specific examples of an additive, include DISPARLON (registered trademark) LS series, LS-430, LS-460, LS-480, and the like, manufactured by Kusumoto Chemicals Ltd., KP series, KP-112, KP-323, KP-326, KP-361, and the like manufactured by Shin-Etsu Chemical Co., Ltd., and TEGO (registered trademark) Glide series, 100, 110, 410, 440, and 482, and TEGO (registered trademark) Phobe series, 1000, 1300, 1505, and 6600 manufactured by TOMOE Engineering Co., Ltd., but not limited thereto.

The amount of the leveling agent added based on 100 parts by mass of the component (A) is, for example, 0.1 to 1.0 part by mass, and is preferably 0.1 to 0.5 parts by mass.

In the present invention, a coupling agent can be added as long as features of the present invention are not impaired. Examples of the coupling agent include a silane-based coupling agent having an epoxy group, a vinyl group, an acryloyl group or a methacryloyl group, and also a hydrolyzable silane group in combination, a polyorganosiloxane having a phenyl group and a hydrolyzable silyl group, and/or a polyorganosiloxane having an epoxy group and a hydrolyzable silyl group, but not limited thereto. Specific examples of the silane-based coupling agent include allyltrimethoxysilane, vinyltrimethoxysilane, 3-glycidoxypropyltrimethoxysilane, 3-methacryloxypropyltrimethoxysilane, 3-acryloxypropyltrimethoxysilane, and 3-chloropropyltrimethoxysilane, but not limited thereto.

A filling agent such as an inorganic filling agent and an organic filling agent can be added in the photocurable composition of the present invention, as long as features of the present invention are not impaired. Such a filling agent can be added to adjust not only viscous properties/thixotropy, but also curability and toughness. Examples of the inorganic filling agent include alumina, silica, and amorphous silica, but not limited thereto. On the other hand, examples of the organic filling agent include a styrene filler, a rubber filler, and a core-shell acrylic filler, but not limited thereto. Examples of a specific product of silica include FUSELEX E-1 manufactured by Tatsumori Ltd. and AO-802 manufactured by Admafine, and examples of a specific product of amorphous silica include Aerosil series, 200 (not treated), R972 (treated with dimethyldichlorosilane), R976 (treated with dimethyldichlorosilane), RY200 (treated with dimethylsilicone), RX200 (treated with hexamethyldisilazane), and R800 (treated with octylsilane) manufactured by Nippon Aerosil Co., Ltd., but not limited thereto.

Before procedures with the photocurable composition according to the present invention, sanding of the surface of a human nail by a file or the like is performed and then any dust, oil content, water content, and the like are removed by a nail dedicated solvent mainly containing ethanol. In the case of coating with the photocurable composition of the present invention, a coating film having a thickness of 100 to 300 μm before curing is formed by a pencil or a brush. A primer may also be used in advance in the coating. A commercially available UV lamp or LED lamp is used as the irradiation apparatus in curing. The irradiation time is 15 seconds to 120 seconds, and is preferably 20 to 70 seconds in consideration of the influence on a finger.

A compound having a (meth)acryloyl group is inhibited from being polymerized by the action of oxygen inhibition in a region where the compound is in touch with oxygen. The photocurable composition according to the present invention is unlikely to be affected by oxygen inhibition and has rapid curability by light irradiation, and thus is suitable for a topcoat for a nail or an artificial nail.

### EXAMPLES

Next, the present invention is further specifically described with reference to Examples, but the present invention is not limited only to these Examples.

### Examples 1 to 5 and Comparative Examples 1 to 7

The following components were provided for preparing a photocurable composition (hereinafter, the photocurable composition being also simply referred to as “composition”.)

### Component (A): Compound Having a (Meth)Acryloyl Group

Polyether-based urethane acrylate oligomer having a weight average molecular weight of 5000 and the number of functional groups of 3 (KY-11 manufactured by Negami Chemical Industrial Co., Ltd.)
Urethane acrylate oligomer having a weight average molecular weight of 4900 and the number of functional groups of 10 (UN-904 manufactured by Negami Chemical Industrial Co., Ltd.)
Urethane acrylate oligomer having a weight average molecular weight of 1500 and the number of functional groups of 6 (UN-3320HA manufactured by Negami Chemical Industrial Co., Ltd.)
Polycarbonate-based urethane acrylate oligomer having a weight average molecular weight of 4500 and the number of functional groups of 2 (UF-8001G, Kyoeisha Chemical Co., Ltd.)
Polyether-based urethane acrylate oligomer having a weight average molecular weight of 5500 and the number of functional groups of 2 (KY-103 manufactured by Negami Chemical Industrial Co., Ltd.)
Tricyclodecane dimethanol diacrylate (Light Acrylate DCP-A manufactured by Kyoeisha Chemical Co., Ltd.)
2-Hydroxypropyl methacrylate (HPMA manufactured by Nippon Shokubai Co., Ltd.)

### Component (B): Polythiol Compound

Trimethylolpropane tris(3-mercaptopropionate) (TMMP-20P manufactured by SC Organic Chemical Co., Ltd.)

### Component (C): A Photoinitiator

1-Hydroxycyclohexyl phenyl ketone (non-visible light type photoinitiator) (IRGACURE 184, manufactured by BASF SE)
2,4,6-Trimethylbenzoyl-diphenyl-phosphine oxide (visible light type photoinitiator) (LUCIRIN TPO manufactured by BASF SE)

### Component (D): Phenol Compound Having One Phenol Group in its Molecule, Represented by General Formula 1

Hereinafter, “A” represents the number of phenols in a molecule and “B” represents the number of organic groups conjugated with a phenol aromatic ring.
Hydroxybenzophenone-based ultraviolet absorber having a molecular weight of 326, and satisfying A: 1 and B: 1 (Adk Stab 1413 manufactured by ADEKA Corporation)
Triazine-based ultraviolet absorber having a molecular weight of 512, and satisfying A: 1 and B: 1 (Adk Stab LA-46 manufactured by ADEKA Corporation)
Benzotriazole-based ultraviolet absorber having a molecular weight of 323, and satisfying A: 1 and B: 1 (Adk Stab LA-29 manufactured by ADEKA Corporation)
Benzotriazole-based ultraviolet absorber having a molecular weight of 225, and satisfying A: 1 and B: 1 (Adk Stab LA-32 manufactured by ADEKA Corporation)
Benzotriazole-based ultraviolet absorber having a molecular weight of 315, and satisfying A: 1 and B: 1 (Adk Stab LA-36 manufactured by ADEKA Corporation)
   Component (D′): Ultraviolet Absorber Other than Component (D)
Hindered amine-based light stabilizer having a molecular weight of 239, and satisfying A: 0 and B: 0 (Adk Stab LA-82 manufactured by ADEKA Corporation)
Hindered amine-based light stabilizer having a molecular weight of 2000, and satisfying A: 0 and B: 0 (Adk Stab LA-63P manufactured by ADEKA Corporation)
Hindered amine-based light stabilizer having a molecular weight of 225, and satisfying A: 0 and B: 0 (Adk Stab LA-87 manufactured by ADEKA Corporation)
Benzotriazole-based ultraviolet absorber having a molecular weight of 659, and satisfying A: 2 and B: 0 (Adk Stab LA-31G manufactured by ADEKA Corporation)
Benzotriazole-based ultraviolet absorber having a molecular weight of 447, and satisfying A: 1 and B: 3 (Adk Stab LA-24 manufactured by ADEKA Corporation)
Triazine-based ultraviolet absorber having a molecular weight of 700, and satisfying A: 3 and B: 0 (Adk Stab LA-F70 manufactured by ADEKA Corporation)

### Other

Polyether-modified silicone (LS-480 manufactured by Kusumoto Chemicals Ltd.).

The component (A), the component (B), the component (D) (component (D′)) and other were weighed in a stirring oven, and stirred for 30 minutes with defoaming in vacuum, to obtain a composition. If the component (D) (or component (D′)) was a solid, this time was elongated until dissolution was achieved. Finally, the component (C) was weighed and added to the stirring oven, and the resultant was stirred for 30 minutes. The detailed amounts of preparation are as shown in Table 1, and all numerical values are expressed by “part(s) by mass”. The amount (by mass) of the component (D) (component (D′)) relative to the entire composition is also shown in Table 1.

TABLE 1

| | | | | | | | Comparative |
|---|---|---|---|---|---|---|---|
| Component | Raw material | Example 1 | Example 2 | Example 3 | Example 4 | Example 5 | Example 1 |
| Component (A) | KY-11 | 60 | 60 | 60 | 60 | 60 | 60 |
| | DCP-A | 10 | 10 | 10 | 10 | 10 | 10 |
| | HPMA | 30 | 30 | 30 | 30 | 30 | 30 |
| Component (B) | TMMP-20P | 17.4 | 17.4 | 17.4 | 17.4 | 17.4 | 17.4 |
| Component (C) | 184 | 3.5 | 3.5 | 3.5 | 3.5 | 3.5 | 3.5 |
| | TPO | 3 | 3 | 3 | 3 | 3 | 3 |
| Component (D) | 1413 | 0.6 | | | | | |
| | LA-46 | | 0.6 | | | | |
| | LA-29 | | | 0.6 | | | |
| | LA-32 | | | | 0.6 | | |
| | LA-36 | | | | | 0.6 | |
| Component (D′) | LA-82 | | | | | | |
| | LA-63P | | | | | | |
| | LA-87 | | | | | | |
| | LA-31G | | | | | | |
| | LA-24 | | | | | | |
| | LA-F70 | | | | | | |
| Other | LS-480 | 0.3 | 0.3 | 0.3 | 0.3 | 0.3 | 0.3 |

| | | | | | | |
|---|---|---|---|---|---|---|
| Total | 124.76 | 124.76 | 124.76 | 124.76 | 124.76 | 124.16 |
| Amount (%) of component (D) (component | 0.48 | 0.48 | 0.48 | 0.48 | 0.48 | 0.00 |
| (D′)) added relative to entire composition | | | | | | |

| | | Comparative | Comparative | Comparative | Comparative | Comparative | Comparative |
|---|---|---|---|---|---|---|---|
| Component | Raw material | Example 2 | Example 3 | Example 4 | Example 5 | Example 6 | Example 7 |
| Component (A) | KY-11 | 60 | 60 | 60 | 60 | 60 | 60 |
| | DCP-A | 10 | 10 | 10 | 10 | 10 | 10 |
| | HPMA | 30 | 30 | 30 | 30 | 30 | 30 |
| Component (B) | TMMP-20P | 17.4 | 17.4 | 17.4 | 17.4 | 17.4 | 17.4 |
| Component (C) | 184 | 3.5 | 3.5 | 3.5 | 3.5 | 3.5 | 3.5 |
| | TPO | 3 | 3 | 3 | 3 | 3 | 3 |
| Component (D) | 1413 | | | | | | |
| | LA-46 | | | | | | |
| | LA-29 | | | | | | |
| | LA -32 | | | | | | |
| | LA-36 | | | | | | |
| Component (D′) | LA-82 | 0.6 | | | | | |
| | LA-63P | | 0.6 | | | | |
| | LA-87 | | | 0.6 | | | |
| | LA-31G | | | | 0.6 | | |
| | LA-24 | | | | | 0.6 | |
| | LA-F70 | | | | | | 0.6 |
| Other | LS-480 | 0.3 | 0.3 | 0.3 | 0.3 | 0.3 | 0.3 |

| | | | | | | |
|---|---|---|---|---|---|---|
| Total | 124.76 | 124.76 | 124.76 | 124.76 | 124.76 | 124.76 |
| Amount (%) of component (D) (component | 0.48 | 0.48 | 0.48 | 0.48 | 0.48 | 0.48 |
| (D′) added relative to entire composition | | | | | | |

In Examples 1 to 5 and Comparative Examples 1 to 7, the solubility was confirmed, the storage stability was confirmed, the photocurability was confirmed, the heat generation during procedures was confirmed and the differential scanning calorimeter (Photo-DSC) measurement was carried out. The results are summarized in Table 2.

### [Confirmation of Solubility]

HPMA and the component (D) (or component (D′)) were mixed at a mass ratio of 100:1 in a glass container, lidded, and left to still stand and heated in a hot air drying furnace under an atmosphere at 60° C. “Solubility” was determined by visual observation according to the following evaluation criteria. In consideration of the appearance, “Good” is preferred. Comparative Example 1 did not include the component (D) (or component (D′)) and thus “-” was described.

### Evaluation Criteria

Good: dissolved (uniform and transparent liquid)
Poor: not dissolved (solid remaining on glass container bottom).

### [Confirmation of Storage Stability]

The composition was placed in an ointment container, left to still stand in a constant-temperature bath set under an atmosphere at 25° C., and left to still stand at an interval of 10 days, 20 days, or 30 days, and the state was visually confirmed according to the following evaluation criteria. In Comparative Examples 5 to 7, the solubility was evaluated as “Poor”, and thus the storage stability was not evaluated and “-” was described.

### Evaluation Criteria

Good: fluidity exhibited even after 30 days at 25° C.
Poor: abnormality observed within 29 days at 25° C. (cured, or gel formed).

### [Confirmation of Photocurability]

An acrylic plate of 2.0 mm thickness×25 mm width×100 mm length was coated with the composition by a brush at a thickness of about 300 μm. The composition was cured by irradiation with an UV lamp for nails (rated voltage: 100 to 110 V, consumed power at 50 to 60 Hz: 36 W, wavelength: 350 to 400 nm) for 60 seconds. The surface of a cured product here was touched with a Teflon (registered trademark) bar to confirm whether or not stickiness was exhibited, and “Surface curability (UV lamp)” was determined according to the following evaluation criteria. In a case where the storage stability was evaluated as “Poor”, the storage stability was not evaluated and “-” was described.

### Evaluation Criteria

Good: no bleeding of components onto surface
Poor: bleeding of components onto surface.

### [Confirmation of Heat Generation During Procedures]

After sanding of a nail was carried out, any dust and oil content were removed by a nail dedicated solvent (mainly containing ethanol). The nail was coated with the composition as a basecoat so that the wet thickness was about 300 μm. The coating was performed by a brush. Thereafter, the composition was cured with irradiation by a LED lamp for nails (rated voltage: 240 V, consumed power at 50 to 60 Hz: 30 W, wavelength: 400 to 410 nm) for 30 seconds. The same method was performed to sequentially cure a colorcoat and a topcoat on the surface of the basecoat under the same conditions. The colorcoat used was Super Color EX (color: pastel peach) manufactured by PREGEL, and the topcoat used was VL-00 manufactured by VETRO Co., Ltd. “Heat generation during procedures” was evaluated with respect to fingernails (10 fingers) of the hand of one person, according to the following evaluation criteria. In a case where the storage stability was evaluated as “Poor”, the storage stability was not evaluated and “-” was described.

### Evaluation Criteria

Good: no hotness felt
Fair: slightly warm
Poor: hot.

### [Differential Scanning Calorimeter (Photo-DSC) Measurement]

Weighed was 0.05 mg of the composition in an aluminum pan for measurement. The aluminum pan was put in a measurement section of a differential scanning calorimeter X-DSC7000 manufactured by Hitachi High-Tech Science Corporation. The composition was irradiated with an active energy ray at a wavelength of 365 nm and an illuminance of 3 mW/cm² for 180 seconds (cumulative amount of light: 540 mJ/cm²) by use of a spot ultraviolet irradiator (lamp used: L8252) manufactured by Hamamatsu Photonics K.K., to perform measurement. The measurement was here performed under the assumption that the heat content (enthalpy) of the exothermic peak was “Heat content (mJ/mg)” and the heat flow at the top of the exothermic peak was “Heat flow (mW)”. The heat content is preferably 250 mJ/mg or less, and the heat flow is preferably 10.0 mW or less.

TABLE 2

| | | | | | | | Comparative |
|---|---|---|---|---|---|---|---|
| Test item | | Example 1 | Example 2 | Example 3 | Example 4 | Example 5 | Example 1 |
| Solubility | | Good | Good | Good | Good | Good | — |
| Storage stability | | Good | Good | Good | Good | Good | Good |
| Surface curability | | Good | Good | Good | Good | Good | Good |
| Heat generation during | | Good | Good | Good | Good | Good | Poor |
| procedures | | | | | | | |
| Photo-DSC | Heat content | 248 | 227 | 227 | 220 | 224 | 276 |
| | Heat flow | 9.5 | 7.0 | 5.5 | 6.7 | 5.6 | 12.4 |

| | | Comparative | Comparative | Comparative | Comparative | Comparative | Comparative |
|---|---|---|---|---|---|---|---|
| Test item | | Example 2 | Example 3 | Example 4 | Example 5 | Example 6 | Example 7 |
| Solubility | | Good | Good | Good | Poor | Poor | Poor |
| Storage stability | | Poor | Poor | Poor | — | — | — |
| Surface curability | | — | — | — | — | — | — |
| Heat generation during | | — | — | — | — | — | — |
| procedures | | | | | | | |
| Photo-DSC | Heat content | — | — | — | — | — | — |
| | Heat flow | — | — | — | — | — | — |

The component (D) used in Examples 1 to 5 was a compound having the number of phenols of 1 and the number of organic groups conjugated with a phenol aromatic ring, of 1, in its molecule, and allowed both storage stability and surface curability to be satisfied. Furthermore, low heat generation in 10 procedures thus caused no hotness felt in fingers in photocuring, and correlated with a small amount of heat of Photo-DSC. On the other hand, each compound not corresponding to the component (D) in the present invention, used in Comparative Examples 5 to 7, was a compound originally inferior in solubility, and no dissolution was made and thus the effect by the corresponding component could not be exerted. Furthermore, Comparative Examples 2 to 4, although achieved dissolution, exhibited inferior storage stability and did not enable both storage stability and surface curability to be achieved.

In each of Reference Examples 1 to 8, the component (A), the component (B), the component (D) and other were weighed in a stirring oven, and then stirred for 30 minutes with defoaming in vacuum. If the component (D) was a solid, this time was elongated until dissolution was achieved. Finally, the component (C) was weighed and added to the stirring oven, and the resultant was stirred for 30 minutes. The detailed amounts of preparation are as shown in Table 3, and all numerical values are expressed by “part(s) by mass”. The amount (% by mass) of the component (D) relative to the entire composition is also shown in Table 3.

TABLE 3

| | | | Compar- | | | | | | | | |
|---|---|---|---|---|---|---|---|---|---|---|---|
| | Raw | Exam- | ative | Reference | Reference | Reference | Reference | Reference | Reference | Reference | Reference |
| Component | material | ple 3 | Example 1 | Example 1 | Example 2 | Example 3 | Example 4 | Example 5 | Example 6 | Example 7 | Example 8 |
| Component (A) | KY-11 | 60 | 60 | | | | | | | | |
| | UN-904 | | | 60 | | | | 60 | | | |
| | UN-3320HA | | | | 60 | | | | 60 | | |
| | UF-8001G | | | | | 60 | | | | 60 | |
| | KY-103 | | | | | | 60 | | | | 60 |
| | DCP-A | 10 | 10 | 10 | 10 | 10 | 10 | 10 | 10 | 10 | 10 |
| | HPMA | 30 | 30 | 30 | 30 | 30 | 30 | 30 | 30 | 30 | 30 |
| Component (B) | TMMP-20P | 17.4 | 17.4 | 17.4 | 17.4 | 17.4 | 17.4 | 17.4 | 17.4 | 17.4 | 17.4 |
| Component (C) | 184 | 3.5 | 3.5 | 3.5 | 3.5 | 3.5 | 3.5 | 3.5 | 3.5 | 3.5 | 3.5 |
| | TPO | 3 | 3 | 3 | 3 | 3 | 3 | 3 | 3 | 3 | 3 |
| Component (D) | LA-29 | 0.6 | | 0.6 | 0.6 | 0.6 | 0.6 | | | | |
| Other | LS-480 | 0.3 | 0.3 | 0.3 | 0.3 | 0.3 | 0.3 | 0.3 | 0.3 | 0.3 | 0.3 |

| | | | | | | | | | | |
|---|---|---|---|---|---|---|---|---|---|---|
| Total | 124.76 | 124.16 | 124.76 | 124.76 | 124.76 | 124.76 | 124.16 | 124.16 | 124.16 | 124.16 |
| Amount (%) of component (D) | 0.48 | 0.00 | 0.48 | 0.48 | 0.48 | 0.48 | 0.00 | 0.00 | 0.00 | 0.00 |
| added relative to entire | | | | | | | | | | |
| composition | | | | | | | | | | |

In Reference Examples 1 to 8, the solubility was confirmed, the storage stability was confirmed, the photocurability was confirmed, the heat generation during procedures was confirmed and the differential scanning calorimeter (Photo-DSC) measurement was carried out. The results are summarized in Table 4.

TABLE 4

| | | | Comparative | Reference | Reference | Reference | Reference | Reference | Reference | Reference | Reference |
|---|---|---|---|---|---|---|---|---|---|---|---|
| Test item | | Example 3 | Example 1 | Example 1 | Example 2 | Example 3 | Example 4 | Example 5 | Example 6 | Example 7 | Example 8 |
| Solubility | | Good | — | Good | Good | Good | Good | — | — | — | — |
| Storage stability | | Good | Good | Good | Good | Good | Good | Good | Good | Good | Good |
| Surface curability | | Good | Good | Good | Good | Poor | Poor | Good | Good | Poor | Poor |
| Heat generation during | | Good | Poor | Poor | Poor | Good | Good | Poor | Poor | Good | Good |
| procedures | | | | | | | | | | | |
| Photo-DSC | Heat content | 227 | 276 | 271 | 265 | 170 | 159 | 291 | 285 | 242 | 227 |
| | Heat flow | 5.5 | 12.4 | 13.7 | 12.2 | 3.1 | 2.8 | 16.2 | 15.4 | 9.1 | 6.1 |

It can be seen that a (meth)acrylate oligomer having 3 to 5 (meth)acryloyl groups in one molecule is used as the component (A) to enhance photocurability and allow heat generation during procedures and properties Photo-DSC to be good. It is noted that use of a (meth)acrylate oligomer having 2, or 6 or more (meth)acryloyl groups in one molecule is not excluded.

### INDUSTRIAL APPLICABILITY

The present invention relates to a photocurable composition which, although contains polythiol, is suppressed in heat generation when photocured, and which has both surface curability and storage stability. In particular, heat generation is suppressed in procedures in the nail field to enable any burden on a finger to be reduced and enable stable procedures to be made, and particularly allow for use for a topcoat.

The present application is based on Japanese Patent Application No. 2021-184528 filed on Nov. 12, 2021, the disclosure of which is incorporated by reference in its entirety.

1. A photocurable composition comprising component (A) to component (D):
Component (A): a compound having a (meth)acryloyl group;
Component (B): a polythiol compound;
Component (C): a photoinitiator; and
Component (D): a phenol compound having one phenol group in its molecule, represented by the following general formula 1, except for the component (A): 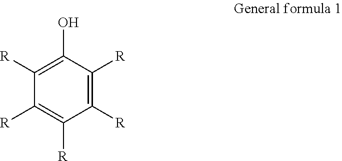
wherein only one among Rs is an organic group conjugated with a phenol aromatic ring, and the remaining Rs are each independently hydrogen or a linear organic group.

2. The photocurable composition according to claim 1, wherein the organic group conjugated with a phenol aromatic ring is a group of formula 2, a group of formula 3 or a group of formula 4, or a group having a backbone of formula 2, formula 3 or formula 4, represented below:

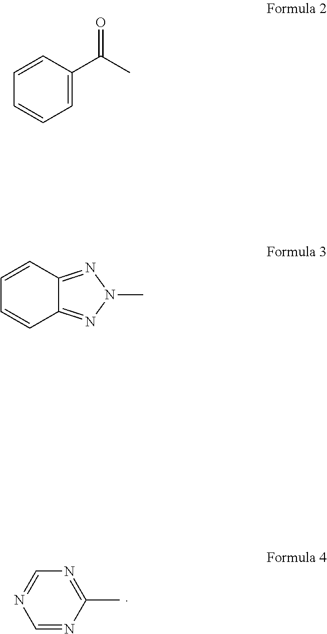

3. The photocurable composition according to claim 1, comprising 0.001 to 10.0% by mass of the component (D) relative to the entire photocurable composition.

4. The photocurable composition according to claim 1, comprising no ultraviolet absorber other than the component (D) in the photocurable composition.

5. The photocurable composition according to claim 1, comprising 0.1 to 50 parts by mass of the component (B) and 0.1 to 10 parts by mass of the component (C) based on 100 parts by mass of the component (A).

6. The photocurable composition according to claim 1, wherein the component (A) is composed of a (meth)acrylate oligomer and a (meth)acrylate monomer.

7. The photocurable composition according to claim 6, comprising a (meth)acrylate oligomer having 3 to 5 (meth)acryloyl groups per molecule, as the (meth)acrylate oligomer.

8. The photocurable composition according to claim 6, wherein the (meth)acrylate monomer contains tricyclodecane dimethanol di(meth)acrylate.

9. The photocurable composition according to claim 1, for use in a nail or an artificial nail.

10. The photocurable composition according to claim 9, wherein the composition is for a topcoat.
